(19) United States
(12) Patent Application Publication
(10) Pub. No.: US 2012/0046713 A1
(43) Pub. Date: Feb. 23, 2012

(54) HEARING AID AND/OR TINNITUS THERAPY DEVICE

(75) Inventors: RONNY HANNEMANN, BUCKENHOF (DE); WINFRIED SCHLEE, SCHWEBHEIM (DE); NADINE SEUBERT, ERLANGEN (DE); MARTINA ZIEGLER, BUCKENHOF (DE)

(73) Assignee: SIEMENS MEDICAL INSTRUMENTS PTE. LTD., SINGAPORE (SG)

(21) Appl. No.: 13/214,448

(22) Filed: Aug. 22, 2011

(30) Foreign Application Priority Data
Aug. 20, 2010 (DE) 10 2010 039 589.7

(51) Int. Cl. A61F 11/04

(52) U.S. Cl. 607/57

(57) ABSTRACT

While prior art hearing aids and/or tinnitus therapy devices only contribute to making an existing tinnitus condition more bearable, the invention provides for a method for operating a hearing aid and/or tinnitus therapy device as well as a hearing aid and/or tinnitus therapy device which contributes to a tinnitus being able to be eliminated over the long-term. An acoustic output signal is generated in which the tinnitus frequency and/or the tinnitus frequency spectrum are suppressed. This makes the neuroplastic reorganization of the central auditory system of a sufferer possible, which reverses the tinnitus-causing maladaptive change in their central auditory system.

### CROSS-REFERENCE TO RELATED APPLICATION

This application claims the priority, under 35 U.S.C. §119, of German patent application DE 10 2010 039 589.7, filed Aug. 20, 2010; the prior application is herewith incorporated by reference in its entirety.

### BACKGROUND OF THE INVENTION

### Field of the Invention

The invention relates to the operation of a hearing aid and/or tinnitus therapy device, whereby at least one tinnitus frequency and/or a tinnitus frequency spectrum of a user is recorded and an acoustic output signal is created as a function of the tinnitus frequency and/or a tinnitus frequency spectrum of the user.

The invention further relates to a hearing aid and/or tinnitus therapy device for carrying out the method.

The problem of chronic tinnitus affects around 10-15% of the western population. In 1-2% of the population the tinnitus leads to severe psychological problems, affects a person's professional and social life, disturbs concentration, causes cognitive and emotional suffering and frequently leads to insomnia.

In many cases chronic tinnitus is accompanied by a severe loss of hearing. A severe loss of hearing is also frequently the trigger for a neuroplastic reorganization of the central auditory system and thus frequently the trigger and the actual cause of the tinnitus.

Electronic aids, so-called tinnitus maskers, are known that create an acoustic signal of which the frequency approximately corresponds to the tinnitus frequency and thus is designed to cover over (mask out) the tinnitus by an acoustic signal supplied from outside to the hearing of a sufferer.

A programmable hearing aid is known from commonly assigned U.S. Pat. No. 6,047,074 and its counterpart European published patent application EP 0 820 211 A1 which is also able to be used for tinnitus therapy as well as for retraining therapy. The digital hearing aid has a signal processor comprising means for signal generation, of which the signals are able to be combined with a digitized user signal.

A method for tinnitus therapy is known from a publication by Hidehiko Okamotoa, Henning Strackea, Wolfgang Sollb, Christo Panteva: Listening to tailor-made notched music reduces tinnitus loudness and tinnitus-related auditory cortex activity. In PNAS, vol 107, no. 3, 19 Jan. 2010, p. 1207-1210, in which a user of the device is provided with an acoustic signal, e.g. music in the manner in which at least one tinnitus frequency or a tinnitus frequency is suppressed.

The disadvantage of the known devices able to be used when a tinnitus noise (tinnitus) is present is that these only make the tinnitus bearable while they are being used but do not contribute to a lasting remedy for the problem.

### SUMMARY OF THE INVENTION

It is accordingly an object of the invention to provide a hearing device which overcome the above-mentioned disadvantages of the heretofore-known devices and methods of this general type and which provides for a method for operating a hearing aid and/or tinnitus therapy device as well as a hearing aid and/or tinnitus therapy device through which the tinnitus can be removed over the long term.

With the foregoing and other objects in view there is provided, in accordance with the invention, a method of operating a hearing device (i.e., a hearing aid and/or tinnitus therapy device), the method which comprises the following steps:

recording at least one tinnitus frequency and/or a tinnitus frequency spectrum of a user; and

generating an acoustic output signal in which the tinnitus frequency and/or the tinnitus frequency spectrum are suppressed.

Further, a hearing device (e.g., a combined hearing aid and tinnitus therapy device) for carrying out the novel method comprises:

at least one signal processor for generating an electric output signal;

an electro-acoustic output converter connected to the signal processor and configured for converting the electric output signal into an acoustic output signal; and means (e.g., a filter, filter bank) for suppressing a tinnitus frequency and/or a tinnitus frequency spectrum in the acoustic output signal.

In other words, the objects are achieved, in accordance with the invention, with a novel method for operating a hearing aid and/or tinnitus therapy device in which at least one tinnitus frequency and/or a tinnitus frequency spectrum of a user is initially recorded. The frequency or the frequencies are established at which the sufferer hears acoustic signals that are not caused by acoustic signals reaching the ear from the outside. This can be done by a doctor or by an audiologist. The hearing aid and/or tinnitus therapy device for supplying the sufferer is then set so that an acoustic output signal is created in which the tinnitus frequency and/or the tinnitus frequency spectrum are suppressed. Unlike known devices employed for tinnitus, the tinnitus frequency or the tinnitus frequency range is thus suppressed here. This makes the neuroplastic reorganization of the central auditory system of a sufferer possible, which reverses the maladaptive neuroplastic reorganization of the central audio system of a sufferer which causes tinnitus.

Advantageously the hearing device, that is, the hearing aid and/or tinnitus therapy device, is embodied for closed supply in which either the device itself or an otoplastic connected to it closes off the auditory canal of the sufferer as well as possible when the device is being worn, so that only the acoustic output signal generated by the device is essentially supplied to the sufferer's hearing. However the inventive masking can also be very effective for an open adaptation.

In a form of embodiment of the invention in which a tinnitus masking signal is actively generated by the hearing aid and/or tinnitus therapy device, the signal is generated such that the tinnitus frequency and/or the tinnitus frequency spectrum are suppressed in the tinnitus masking signal issued by the hearing aid and/or tinnitus therapy device. This can typically be achieved by a signal generator available for signal generation not generating the frequencies or frequency ranges involved in the masking signal at all or by the tinnitus frequency concerned or the tinnitus frequency spectrum concerned being filtered out afterwards from the originally generated masking signal (suppressed). Notch filters or bandpass filters can be used as filter means in such cases. Preferably not only are the tinnitus frequency or the tinnitus frequency spectrum suppressed by such means but also a specific bandwidth around the tinnitus frequency or the tinnitus frequency spectrum. Preferably, in addition to the actual filter frequency or the actual filter frequency band, this bandwidth can also be set by programming the relevant hearing aid and/or tinnitus therapy device.

Since a tinnitus is frequently accompanied by a hearing loss of the sufferer, an inventive device preferably serves both for tinnitus therapy and also to compensate for the hearing loss. To this end an acoustic input signal is picked up in the hearing aid and tinnitus therapy device and processed therein and in particular amplified depending on frequency to compensate for a user's individual hearing loss. The acoustic output signal thus includes a signal originating from the acoustic input signal. In addition the acoustic output signal can however also comprise a masking signal generated in the hearing aid and tinnitus therapy device. In accordance with the invention, in the combined hearing aid and tinnitus therapy device, the tinnitus frequency and/or the tinnitus frequency spectrum are also suppressed in the generated acoustic output signal.

Other features which are considered as characteristic for the invention are set forth in the appended claims.

Although the invention is illustrated and described herein as embodied in a hearing aid and/or tinnitus therapy device, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 a block diagram of an exemplary embodiment of the novel hearing aid and tinnitus therapy device;

FIG. 2 a flowchart illustrating the execution of the novel method; and

FIG. 3 a block diagram of a further exemplary embodiment of the hearing aid and tinnitus therapy device.

### DETAILED DESCRIPTION OF THE INVENTION

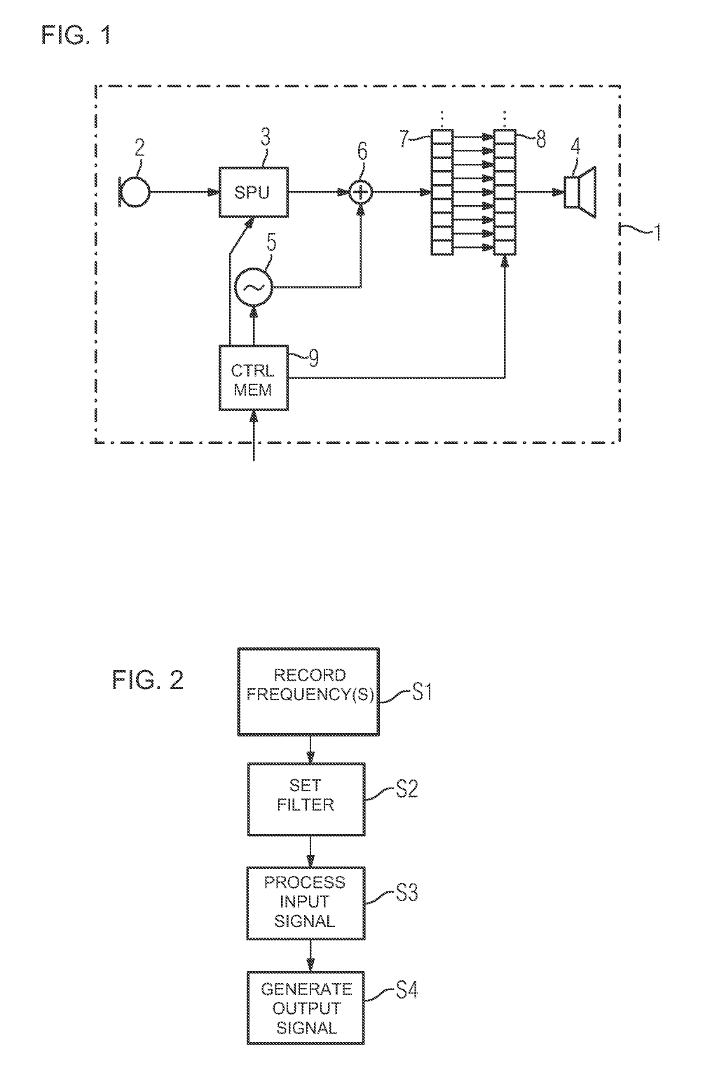

Referring now to the figures of the drawing in detail and first, particularly, to FIG. 1 thereof, there is shown a hearing device 1, here a combined hearing aid and tinnitus therapy device 1, according to the invention. The hearing device comprises an input converter, such as a microphone 2 for picking up an acoustic input signal and converting it into an electric input signal. For further processing, especially for frequency-dependent amplification to compensate for the individual hearing loss of the user, the electric input signal is fed to a signal processing unit 3 for generation of an electric output signal. The electric output signal of the signal processing unit 3 is finally—if necessary after further processing—fed to an electro-acoustic output converter, here in the form of an earpiece 4, for conversion of the electric output signal into an acoustic output signal.

In the hearing aid and tinnitus therapy device 1 in accordance with the exemplary embodiment, a signal processor, in addition to the signal processing unit 3, also includes a signal generator 5 for tinnitus therapy, which especially generates an individual masking signal, i.e. a signal able to be adapted to the individual tinnitus noise of a user. This is added in a summation unit 6 to the electric output signal output by the signal processing unit 3.

The inventive hearing aid and tinnitus therapy device 1 further includes as a filter means a filter bank 7 in which the electric output signal output by the summation unit 6 is split up into a plurality of frequency bands. Subsequently the frequency bands are merged again in a synthesis unit 8 into an electric output signal, especially added, and converted by the earpiece 4 into an acoustic signal and supplied to the hearing of the user.

The inventive hearing aid and tinnitus therapy device 1 also includes a control and memory unit 9 which essentially controls the suppression of the tinnitus frequency and/or of the tinnitus frequency spectrum in the output signal output by the hearing aid and tinnitus therapy device 1. The control and memory unit 9 is able to be connected for this purpose to an external programming device (not shown in the figure). The signal processing unit 3 for individual adaptation of the signal processing to the hearing loss of the user is adjusted via the control and memory unit 9. The adjustment of the signal generator 5 and in this context especially the adjustment of the generated signal frequencies and signal level of the masking signal is also undertaken by said device. Furthermore the control and memory unit 9 determines which frequency bands will be allowed to pass (unhindered) by the synthesis unit and which frequency bands will be suppressed (blocked). A large number (e.g. greater than 100) of frequency bands in the filter bank 7 (multichannel filter bank) and the synthesis unit 8 makes it possible to adapt the acoustic output signal all the more precisely to the individual tinnitus noise. Transfer of the signal processing from the time domain into the frequency domain is also considered, which for digital signal processing corresponds to the use of a large number of frequency bands.

Unlike in the exemplary embodiment shown, the filter bank 7 can also be arranged between the microphone 2 and the signal processing unit 3 (not shown in the figure) so that the signal processing in the signal processing unit 3 is also undertaken in a plurality of frequency bands.

An exemplary embodiment of the method for operating a hearing aid and tinnitus therapy device is illustrated in the flowchart of FIG. 2. It comprises the following steps:
Step S1: Recording at least one tinnitus frequency and/or a tinnitus frequency spectrum of the user;
Step S2: Setting of filter means;
Step S3: Receiving an acoustic input signal into the hearing aid and tinnitus therapy device and processing the received input signal;
Step S4: Generating an acoustic output signal in which the tinnitus frequency and/or the tinnitus frequency spectrum are suppressed.

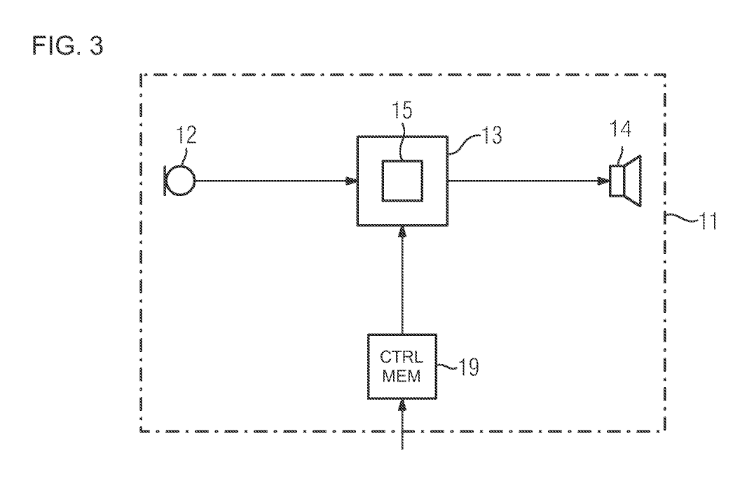

A further exemplary embodiment of a hearing aid and tinnitus therapy device is shown in FIG. 3. The hearing aid and tinnitus therapy device 11 comprises an input converter, especially a microphone 12 for receiving an acoustic input signal and converting it into an electric input signal. For further processing, especially for frequency-dependent amplification to compensate for the individual hearing loss of the user, the electric input signal is fed to a signal processing unit 13 for generation of an electric output signal. The electric output signal of the signal processing unit 13 is finally—if necessary after further processing—fed to an electro-acoustic output converter, here in the form of an earpiece 14, for conversion of the electric output signal into an acoustic output signal.

For performing tinnitus therapy the hearing aid and tinnitus therapy device 11 and especially the signal processing unit 13 include filter means 15 through which at least one tinnitus frequency and/or at least one tinnitus frequency spectrum are suppressed in the output signal generated by the signal processing unit 13. To select at least one tinnitus frequency and/or at least one tinnitus frequency spectrum, a control and memory device 19 is available which is able to be connected to an external programming device (not shown in the figure) and via which the transmission function of the filter means 15 is able to be selected.

The inventive hearing aid and tinnitus therapy device 11 makes it possible to suppress frequencies or frequency ranges present in the acoustic input signal picked up by the microphone 12 so that these are no longer present in the acoustic output signal generated by the earpiece 14. In many users this causes a tinnitus which is initially present to recede over time. In an especially preferable embodiment of the hearing device 11, the same is provided for closed supply. This means that the hearing of the user is supplied, at least substantially, only with the acoustic signal generated by the earpiece 14.

1. A method of operating a hearing device, the method which comprises the following steps:
recording at least one tinnitus frequency and/or a tinnitus frequency spectrum of a user; and
generating an acoustic output signal in which the tinnitus frequency and/or the tinnitus frequency spectrum are suppressed.

2. The method according to claim 1, wherein the generating step comprises generating a tinnitus-masking signal such that the tinnitus frequency and/or the tinnitus frequency spectrum are suppressed in the tinnitus-masking signal.

3. The method according to claim 1, which comprises receiving an acoustic input signal in the hearing device, processing the acoustic input signal in the hearing device, generating the acoustic output signal as a function of the acoustic input signal, and thereby suppressing signal components of the acoustic input signal at the tinnitus frequency and/or the tinnitus frequency spectrum in the acoustic output signal.

4. The method according to claim 1, which comprises setting filter means to suppress the tinnitus frequency and/or the tinnitus frequency spectrum in the acoustic output signal.

5. The method according to claim 1, which comprises operating the hearing device as a combined hearing aid and tinnitus therapy device.

6. A hearing device for carrying out the method according to claim 1, comprising:
at least one signal processor for generating an electric output signal;
an electro-acoustic output converter connected to said signal processor for converting the electric output signal into an acoustic output signal; and
means for suppressing a tinnitus frequency and/or a tinnitus frequency spectrum in the acoustic output signal.

7. The hearing device according to claim 6 configured as a combined hearing aid and tinnitus therapy device.

8. The hearing device according to claim 6, which further comprises a filter for suppressing the tinnitus frequency and/or the tinnitus frequency spectrum in the acoustic output signal.

9. The hearing device according to claim 6, wherein said filter means are configured for adjustment by programming the hearing aid and/or tinnitus therapy device.

10. The hearing device according to claim 6, wherein said filter means comprise a multichannel filter bank.

11. A hearing aid and/or tinnitus therapy device configured for a closed supply.
